(19) United States
(12) Patent Application Publication
(10) Pub. No.: US 2009/0131522 A1
(43) Pub. Date: May 21, 2009

(54) Skin Cell Activator Extracted From Liver Of Fish Or Shellfish And Hair Growth Agent Using The Same

(75) Inventors: Yuto Kamei, Saga (JP); Yoshihisa Ota, Saga (JP)

Correspondence Address: JORDAN AND HAMBURG LLP, 122 EAST 42ND STREET, SUITE 4000, NEW YORK, NY, 10168, US

(73) Assignee: MANBOU CORP., Karatsu-shi, Saga (JP)

(21) Appl. No.: 11/921,200

(22) PCT Filed: Jun. 20, 2005
(86) PCT No.: PCT/JP2005/011276

§ 371 Date: Nov. 28, 2007

(30) Foreign Application Priority Data
May 30, 2005 (JP) 2005-156915

(51) Int. Cl. A61K 31/20; A61K 31/045

(52) U.S. Cl. 514/558; 514/724

(57) ABSTRACT

Disclosed are a skin cell activator extracted from a pufferfish liver, and a hair growth agent comprising 10 volume % or more of the skin cell activator. The skin cell activator comprises an organic substance which contains an alcohol- and other organic solvent-soluble fat/oil component extracted from a pufferfish liver, or a high-polar organic substance which has the fat/oil component and a hydrophilic reactive group selected from a group including a carboxyl group and a hydroxyl group and bound to the fat/oil component. The present invention makes it possible to effectively utilize an internal organ of a fish or shellfish which has conventionally been precluded from utilization. In particular, based on clarification of the hair growth-promoting function of the pufferfish's internal organ, the present invention can provide a novel hair growth agent having a direct hair-growing effect by utilizing the function.

### TECHNICAL FIELD

The present invention relates to a skin cell activator which comprises an extract from the liver of a certain type of fish or shellfish, and which has the function of activating human skin cells, as well as a hair growth agent using the skin cell activator.

### BACKGROUND ART

In recent years, the aquafarming of various types of valuable fishes and shellfishes has shown rapid expansion. In connection with this expansion, the disposal of fish and shellfish parts that have no food value, such as internal organs removed during cooking/processing, is emerging as a social issue.

Particularly for toxic fish, as typified by the pufferfish (i.e., blowfish, globefish or fugu), the serious risk in disposal of its internal organs, including a liver containing tetrodotoxin with severe toxicity, has spurred various researches to investigate the effective utilization of the internal organs.

There have also been attempts to utilize an oil component, obtained from a part such as an internal organ of a certain type of fish or shellfish, as a skin agent for external use, such as for hair nourishment or as a cosmetic agent, in the form of a mixture with a certain type of plant or seaweed.

For example, the following Patent Publication 1 discloses a hair nourishment agent comprising a mixture of plant oil, such as camellia oil or olive oil, and unpurified squalane extracted from a deep-sea shark.

The following Patent Publication 2 also discloses that an angiotensin-converting enzyme (ACE) inhibitor consisting of one or more of (1) valyl-tyrosine, isoleucyl-tyrosine and a hydrolysate of a fish or shellfish protein containing these oligopeptides, and (2) valyl-prolyl-proline, isoleucyl-prolyl-proline and a lactic-acid fermentation product of skim milk containing these oligopeptides, serves as a hair-growth/nourishment component capable of improving peripheral blood flow to a scalp and exhibits excellent hair-growing/nourishing effects.

Further, the following Patent Publication 3 discloses that a skin agent for external use comprising an oil, such as plant oil or fish oil, containing three types of unsaturated fatty acids consisting of linoleic acid, linolenic acid and y-linolenic acid, and a fatty acid having a carbon number of 20 or more, has a skin-roughness remedial effect, a skin-elasticity recovery effect and a skin anti-aging effect.

Although many hair growth agents containing an oil obtained from a fish or a shellfish, i.e., so-called “fish oil”, have been known as described above, the function of the fish oil effective in activation of human superficial cells has been uncertain. That is, in both cases, the fish oil has been used in a hair growth agent with an anticipation of only a secondary effect, such as an indirect blood flow-promoting action or sebum secretion-inhibiting action.
[Patent Publication 1] JP 2002-241235A
[Patent Publication 2] JP 2002-284647A
[Patent Publication 3] JP 2003-073268A

### DISCLOSURE OF THE INVENTION

### Problem to be Solved by the Invention

In view of the above circumstances, it is an object of the present invention to allow an internal organ of a fish or shellfish which has been precluded from utilization to be effectively utilized as a human superficial cell activator.

It is another object of the present invention to clarify the function of a fish's or shellfish's internal organ which contains tetrodotoxin, as a human cell activator.

It is yet another object of the present invention to clarify the existence of a hair growth promoting function of an internal organ of a pufferfish, and obtain a novel hair growth agent having a direct hair-growing effect by utilizing the function.

### Means for Solving the Problem

A skin cell activator of the present invention comprises a fat/oil component obtained by dissolving the liver of a fish or a shellfish in an organic solvent selected from a group including alcohol, hexane and chloroform, or a high-polar organic substance which has the fat/oil component and a hydrophilic reactive group selected from a group including a carboxyl group and a hydroxyl group and bound to the fat/oil component.

An organic substance extracted from the liver of a pufferfish and other fishes and shellfishes to provide a hair growth-promoting function.

An extracting liquid for use in extracting the above fat/oil component may be a solvent selected from a group including hexane and chloroform and adapted to dissolve a low-polar fat/oil component, as well as a high-polar solvent selected from a group including phosphate buffered saline (PBS) and MeOH.

When the organic substance containing the fat/oil component soluble in an organic solvent selected from a group including alcohol, hexane and chloroform, or the high-polar organic substance which has the fat/oil component and a hydrophilic reactive group selected from a group including a carboxyl group and a hydroxyl group and bound to the fat/oil component, is used as a human skin cell activator solution, particularly as a hair growth or regrowth agent solution, the solution should contain the organic substance in an amount of 1.0 volume % or more to sufficiently obtain intended effects.

A pufferfish liver to be subjected to extraction of the fat/oil component effective as an ingredient of a hair growth agent may be safely detoxified in advance without losing the intended effects.

### EFFECT OF THE INVENTION

As above, in the present invention, the extract obtained from an internal organ of a fish or a shellfish, particularly from the liver of a pufferfish, serves as a superficial cell (i.e., skin cell) activator, and has a significant effectiveness as a hair growth or regrowth agent.

### BEST MODE FOR CARRYING OUT THE INVENTION

An embodiment of the present invention will now be described based on examples.

### EXAMPLE

A human outer root sheath (h ORS) cell proliferation-promoting activity was evaluated based on the liver and testis of a farm-raised tiger puffer (Takifugu rubripe), and then a liver-extract MeOH solution exhibiting a high cell proliferation-promoting activity was subjected to an in-vivo test of hair-growth effect.

### (Evaluation of Human Outer Root Sheath (h ORS) Cell Proliferation-Promoting Activity)

100 microliters of a 0.01% collagen solution was added to each of 96 wells of a well plate, and the well plate was maintained in a static state for 2 hours. Then, each of the wells of the well plate was rinsed with the same quantity of phosphate buffered saline (PBS) 3 times and coated with the PBS.

100 microliters of human outer root sheath (h ORS) cells were seeded into each of the wells of the well plate (i.e., 2×103 cells/well), and cultured for 24 hours for the purpose of cell attachment.

After the culture, the well plate was transferred to a culture medium [1% FBS supplemented, E-RDF medium/DMEM (GIBCO®, Invitrogen Corp.)=1:1] prepared to contain 1% of a test sample (i.e., the pufferfish liver or testis). Then, the test sample was further cultured for 48 hours.

Subsequently, 10 microliters of 5 mg/ml MTT solution (Sigma-Aldrich Co.) were added to each of the wells of the well plate after the culturing, and reacted with the test sample for 4 hours. Then, 100 microliters of 10% sodium dodecyl sulphate (SDS) (with 0.01 M HCl) was added to each of the wells of the well plate to completely solve out a reaction product, and a light absorption value (570 to 655 nm) of the cultured cells was measured using a micro plate reader (Model 450; Bio-Rad Laboratories, Inc.). Then, an h ORS cell proliferation-promoting activity was evaluated by the following formula on the assumption that the negative control is 100%.

Proliferation-promoting activity (%)=(light absorption value of cells cultured by adding the extract from the pufferfish liver or testis)/(light absorption value of cells cultured by adding only the extracting solvent)

The proliferation-promoting activity of the pufferfish liver extract was 122.2% in a 4 times concentrated solution of a PBS/liver extract solution (i.e., an extract solution prepared from the pufferfish liver using PBS), and 128.5% in a MeOH/liver extract solution (i.e., an extract solution prepared from the pufferfish liver using MeOH) having a concentration of 12.5 micrograms/ml. In contrast, respective proliferation-promoting activities of the PBS/testis extract solution (i.e., an extract solution prepared from the pufferfish testis using PBS) and the MeOH/testis extract solution (i.e., an extract solution prepared from the pufferfish testis using MeOH) were about 105% at best.

This result showed that the pufferfish liver extract has a sufficiently-high cell proliferation-promoting activity.

### (In-Vivo Test on Hair Growth Effect)

An in-vivo test of a hair growth effect based on the h ORS cell proliferation-promoting activity was carried out, and the test result was evaluated on the assumption that the negative control obtained by adding only a solvent for dissolving a test sample, i.e., the extract solution, is 100%.

The hair growth effect test was carried out in the following manner.

C3H/He mice were bred for 6 weeks, and freely given a solid food (product of CLEA Japan, Inc) and tap water. Then, the mice were separated into multiple groups, each consisting of three mice per test plot, and preliminary breeding was carried out for 2 weeks.

After the preliminary breeding, hair on the back of each test mouse was carefully shaved by a shaver to form a rectangular shaved region having a length of about 5 cm and a width of about 3 cm, 50 microliters of the test sample were applied to the shaved region every day for 30 days. The concentration of the applied extract solution was set at 2% (weight/volume), and the hair growth effect was evaluated by observing the state when the shaved skin changed from faint pink to gray in response to hair development, and hairs started growing.

Based on the above evaluation process, an h ORS cell proliferation-promoting activity was evaluated for respective PBS/extract and MeOH/extract solutions prepared from the pufferfish liver and testis.

FIGS. 1 to 4 show respective cell proliferation-promoting activities of the PBS/extract and MeOH/extract solutions prepared from the pufferfish liver and testis, wherein the horizontal axis for the PBS/extract solutions and the horizontal axis for the MeOH/extract solutions represent a dilution rate and a concentration, respectively.

As shown in FIG. 1, in the PBS/liver extract solution, the cell proliferation-promoting activity was exhibited at a dilution rate of 8 times or less. Then, in a 4 times concentrated solution, the cell proliferation-promoting activity was increased by up to 122.2%. As shown in FIG. 2, in the MeOH/liver extract solution, the cell proliferation-promoting activity was increased by up to 128.5% at a concentration of 12.5 microgram/ml.

In contrast, as shown in FIGS. 3 to 4, none of the PBS/extract and MeOH/extract solutions prepared from the pufferfish testis could provide any significant h ORS cell proliferation promoting activity.

Further, based on the above test process, the hair growth effect was evaluated by applying the PBS/liver extract solution to the shaved back region of each mouse. As a result, a grayed skin as a symptom of hair development was observed 10 days after the application, and visible hair growth was observed after 15 days. Finally, 20 days after the application, hairs grew to a state equivalent to that before shaving.

As above, it has been verified that, among the extracts from the pufferfish liver and testis, the MeOH/liver extract has a strong hair growth effect in vivo, and therefore can be favorably applied as a hair growth agent.

### INDUSTRIAL APPLICABILITY

Although the above example has been made of a human outer root sheath (h ORS) cell proliferation promoting activity of an extract from a pufferfish liver, it is believed that the cell proliferation promoting effect is available for general skin cells as well as outer root sheath cells.

### BRIEF DESCRIPTION OF DRAWINGS

FIG. 1 is a graph showing the outer root sheath cell proliferation promoting activity of an extract from a pufferfish liver using PBS.

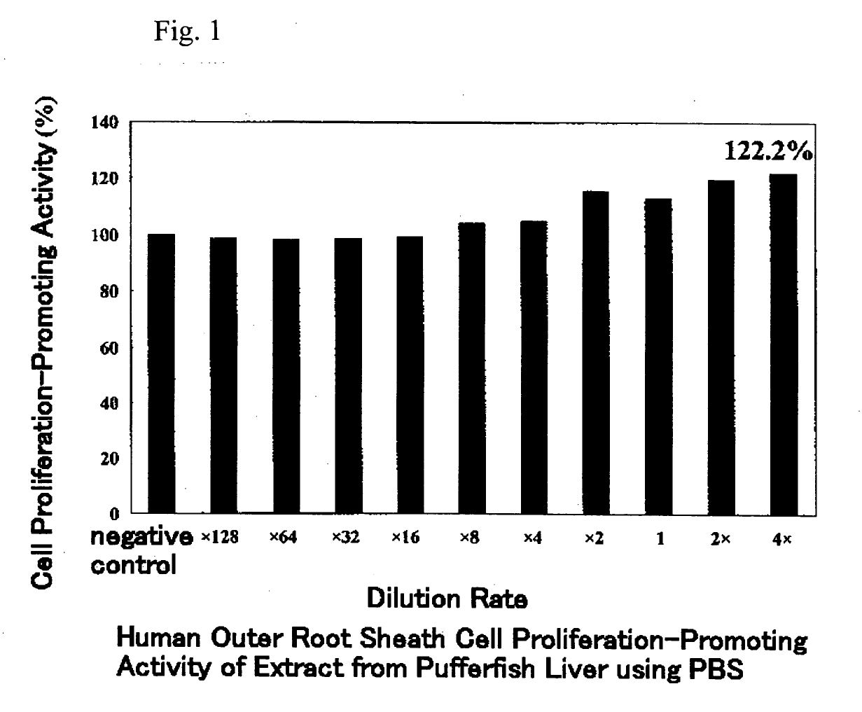

FIG. 2 is a graph showing the outer root sheath cell proliferation promoting activity of an extract from a pufferfish liver using MeOH.

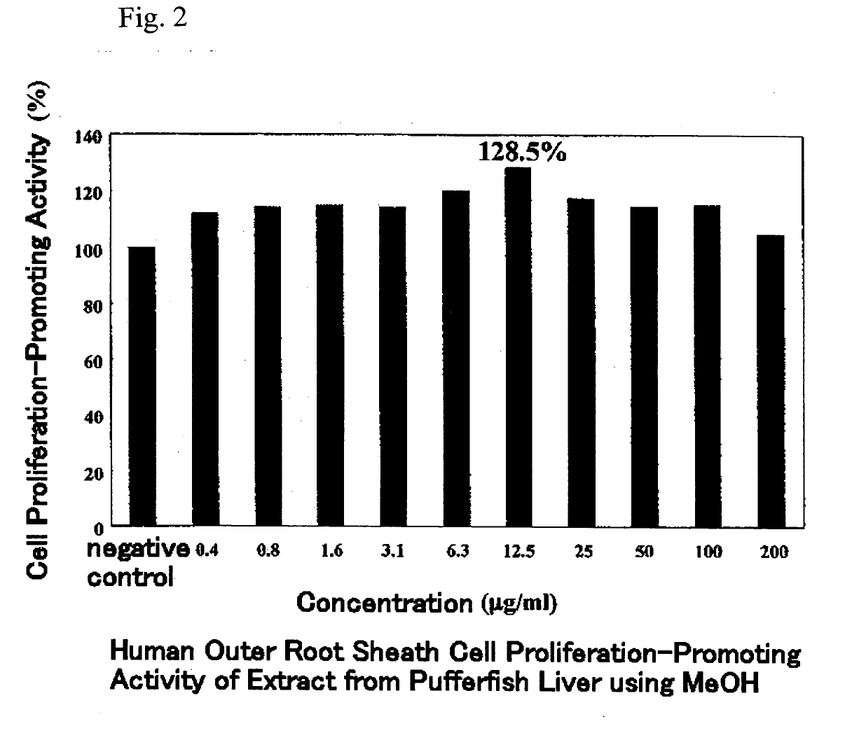

FIG. 3 is a graph showing the outer root sheath cell proliferation promoting activity of an extract from pufferfish testis using PBS.

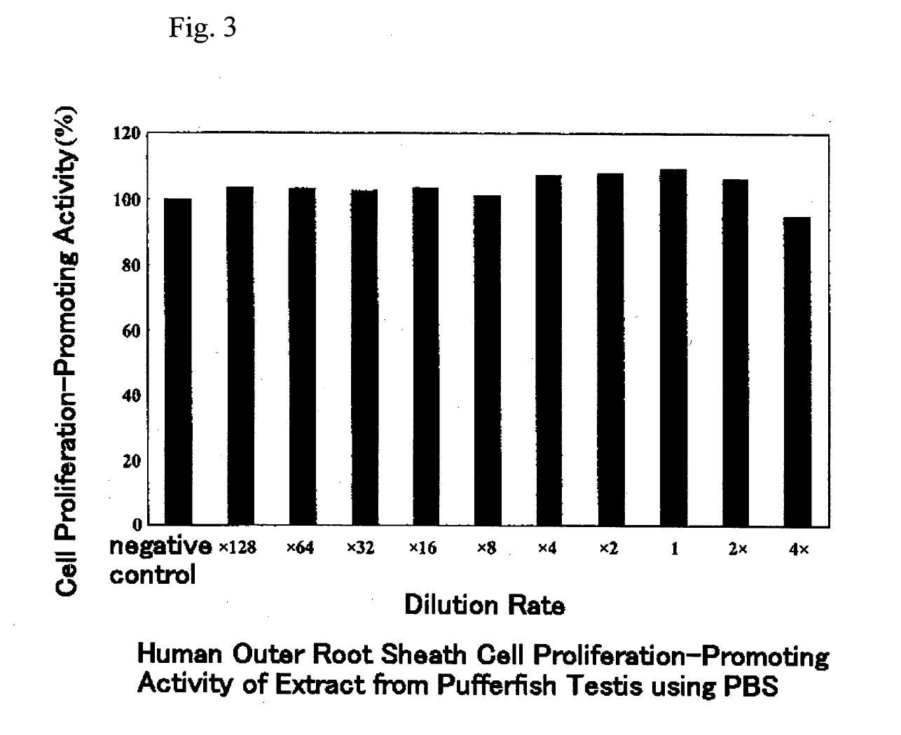

FIG. 4 is a graph showing the outer root sheath cell proliferation promoting activity of an extract from pufferfish testis using MeOH.

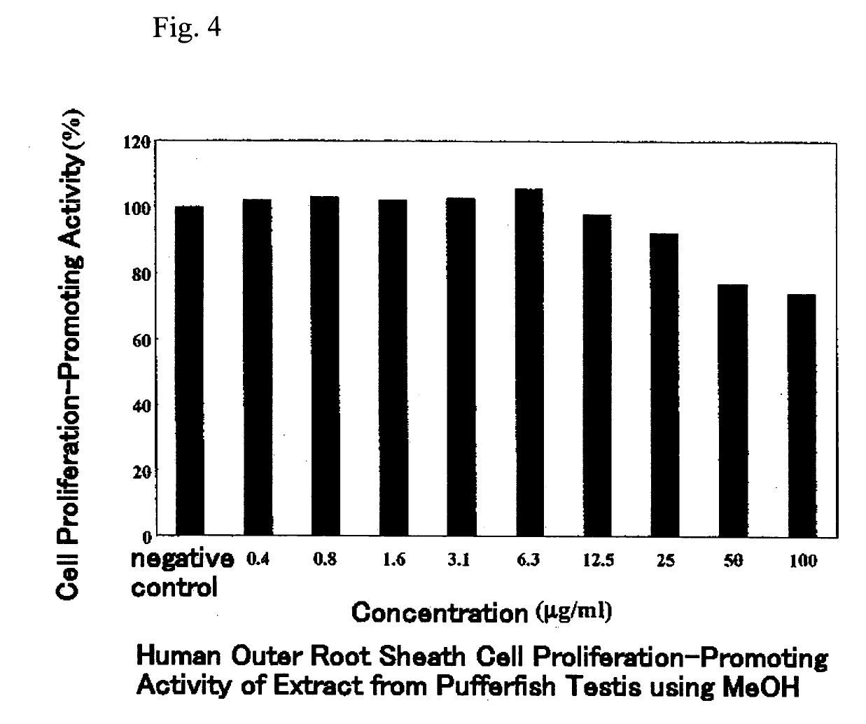

What is claimed is:

1. A skin cell activator comprising an alcohol- and other organic solvent-soluble fat/oil component extracted from a liver of a fish or shellfish using an extracting liquid.

2. A skin cell activator comprising a high-polar organic substance which has an alcohol- and other organic solvent-soluble fat/oil component extracted from a liver of a fish or shellfish using an extracting liquid, and a hydrophilic reactive group selected from a group including a carboxyl group and a hydroxyl group and bound to said fat/oil component.

3. The skin cell activator as defined in claim 1 or 2, wherein said extracting liquid consists of a high-polar solvent selected from a group including phosphate buffered saline (PBS) and MeOH, or a solvent selected from a group including hexane and chloroform and adapted to dissolve a low-polar fat/oil component.

4. The skin cell activator as defined in claim 1 or 2, wherein said fish or shellfish is a pufferfish.

5. A hair growth agent comprising 10 volume % or more of the skin cell activator as defined in claim 1 or 2.
